(19) United States
(12) Patent Application Publication
(10) Pub. No.: US 2012/0238801 A1
(43) Pub. Date: Sep. 20, 2012

(54) SYSTEM FOR AROMATHERAPY IN CONJUNCTION WITH CPAP DEVICE

(76) Inventor: Dale McQueen, Loveland, CO (US)

(21) Appl. No.: 13/424,379

(22) Filed: Mar. 20, 2012

Related U.S. Application Data
(60) Provisional application No. 61/454,558, filed on Mar. 20, 2011.

(51) Int. Cl. A61M 21/02; A61M 16/10

(52) U.S. Cl. 600/27; 128/204.18

(57) ABSTRACT

The present invention provides a system for delivering aromatherapy to a user of a continuous positive airway pressure (CPAP) machine. The system may include a ventilator device, a vessel, and a coupler. The vessel may hold an aromatic substance and may be attached to the ventilator device by the coupler.

### CROSS-REFERENCE TO RELATED APPLICATION

This application claims the benefit of U.S. Provisional Application No. 61/454,558, filed March 20, 2011

### BACKGROUND

Sleep Apnea is an over-relaxation of the soft tissue in the back of the throat that causes a cessation of breathing during sleep states. The breathing cessation can cause a lack of oxygen resulting in drowsiness and sleep disorders during waking hours. Approximately twenty million people in the United States have been diagnosed with Sleep Apnea.

One treatment for Sleep Apnea is a continuous positive airway pressure (CPAP) machine, which provides a continuous flow of air through the nostrils via a mask. The flow of air works to maintain an open airway. CPAP treatment provides pressurized air or other breathable gas to the entrance of a person's airways at a pressure elevated above atmospheric pressure. The pressurized air supplied to the person effectively assists the muscles to keep the person's airway open. Sleep Apnea sufferers must wear the mask for the entire night. Some CPAP users have difficulty sleeping for a week or more as they try to adjust to the use of the apparatus. Most individuals adapt within a month, however, many CPAP users are never completely comfortable and lose hope of relief.

One disadvantage of existing CPAP machines is that they do not function properly if the mask is not fitted properly throughout the night. Claustrophobia and the natural reaction to remove the mask are common among users. This is an issue as many users move in their sleep and remove the mask unconsciously. Furthermore, the moist air that is provided to the user is often stale or even has an unpleasant smell and/or taste.

Aromatherapy has been used to relieve distress attributed to the use of CPAP devices. Scented masks are available, but the scent is strong initially and fades over short-term use. Scented oils are also used but present their own problems as their application is more difficult and introduces cleanliness concerns. Ease of use and consistency of result is a problem with both of these common approaches. Furthermore, the availability and effectiveness varies from one CPAP manufacture to another. Replacement of devices with either of these approaches is complex, often beyond the ability of a large percentage of CPAP users.

A system for implementing aromatherapy in conjunction with a CPAP device is needed. More specifically, a consistent easy to use aromatherapy device is needed in the art.

### SUMMARY

The present invention provides a system for delivering aromatherapy to a user of a continuous positive airway pressure (CPAP) machine. The system may include a ventilator device, a vessel, and a coupler. The vessel may hold an aromatic substance and may be attached to the ventilator device by the coupler.

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter.

### BRIEF DESCRIPTION OF THE DRAWINGS

In the drawings,

FIG. 1 is a schematic diagram of a preferred embodiment of the present invention;

FIG. 2 is a more detailed schematic diagram of the preferred embodiment of the present invention;

FIG. 3 is a schematic diagram of another embodiment of the present invention; and

FIG. 4 is a schematic diagram of one embodiment of a device of the present invention.

### DETAILED DESCRIPTION

An aromatherapy pouch may be designed for use with continuous positive airway pressure (CPAP) machines to reduce or eliminate internal machine odors and promote calm and restful sleep. The aromatherapy pouch may provide scent introduction to a user continuously to aid the user to sleep. In one embodiment, the aromatherapy pouch may be a 3×4 inch pouch made of breathable material. The aromatherapy pouch may use lavender, chamomile, jasmine and/or mint. The scent provided by lavender, chamomile, jasmine, and mint have been known to reduce stress and tension as they are proven aids in relaxation.

CPAP machines have an accessible air intake port. In some embodiments, the aromatherapy pouch may be fastened with string (or tape) to the air intake of a CPAP machine. In other embodiments, a mechanical mechanism may be used to connect the pouch to the air intake.

In one example, a user may take an aromatherapy pouch containing an aromatic substance (e.g., lavender) and attach the pouch to an air intake port of a CPAP machine. In some embodiments, the pouch may be made of organza, nylon, or other breathable mesh materials.

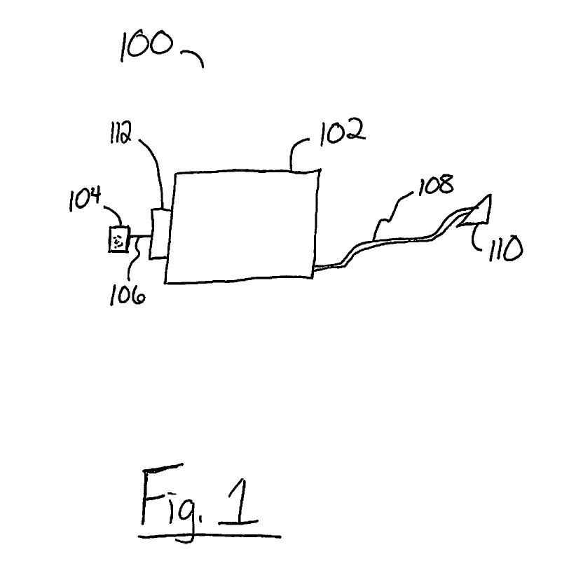

Referring to FIG. 1, a detailed diagram of an apparatus 100 is shown. The apparatus 100 generally comprises a device 102, a vessel 104 and a device 106. The device 102 may be a ventilator device (e.g. a CPAP machine). The ventilator device 102 may be any known ventilator machine on the market (e.g., continuous positive airway pressure (CPAP), piston positive airway pressure, automatic positive airway pressure, variable positive airway pressure, bi-level positive airway pressure, spontaneous time positive airway pressure, and non-invasive positive pressure ventilation ventilators, etc.). The vessel 104 may be a pouch (or bag). The pouch 104 may be configured to hold (or contain) one or more objects (e.g., aromatic substances, aromatherapy products, etc.). The device 106 may be a coupling.

The coupling 106 may be configured to attach the pouch 104 to the CPAP machine 102. The coupling 106 may further be configured to be permanently fixed to the CPAP machine 102. In another example, the coupling 106 may be configured to be permanently fixed to the pouch 104. In yet another example, the coupling 106 may be configured to be freestanding.

The CPAP machine 102 may further comprise a tube 108, an interface device 110, and an air intake 112. The tube 108 may be any standard tubing used in the art. The tube 108 may be configured to be connected to the CPAP machine 102 by any standard means known in the art. The interface device 110 may be a mask, mouthpiece, and/or nasal tubing. The interface device 110 may be configured to connect to the tube 108 by any standard mechanism known in the art. The interface device 110 may be any apparatus or mechanism for delivering gas (e.g., oxygen) to the respiratory system of a subject. The air intake 112 (or air intake port) may generally be found on any standard CPAP machine.

In a preferred embodiment, the pouch 104 may be attached to the CPAP machine 102 at the air intake 112. The coupling 106 may attach the pouch 104 to the air intake 112. Depending on the implementation, the coupling 106 may use one of a plurality of mechanisms (e.g., threads, tape, clips, hooks, hinges, compression fittings, friction fittings, soldering, melting, adhesives, pop-up clips, etc.) to attach the pouch 104 to the air intake 112. It is known that there are many mechanisms by which the coupling 106 may attach the pouch 104 to the CPAP machine 102. Let it be known that any one of a plurality of mechanisms may be used by the coupling 106 to attach the pouch 104 to the CPAP machine 102.

In one example, the coupling 106 may be part of the pouch 104. In this example, the pouch 104 and coupling 106 may be combined into one unit (or device).

The pouch 104 may be made from a breathable (or porous) material (e.g., organza, nylon, cotton, etc.). The coupling 106 may be made from the same material as the pouch 104. However, the coupling 106 may be made from one of a plurality of materials (e.g., organza, nylon, cotton, etc.). The pouch 104 and the coupling 106 may be made from different materials.

The one or more objects generally comprise one or more of a plurality of substances (e.g., aromatic, aromatherapy products, etc.). For example, the one or more objects may be used for medicinal purposes. Aromatic substance may include, but is not limited to, lavender, jasmine, mint and chamomile. The aromatic substance may consist of a mixture of the materials (e.g., ¾ lavender and ¼ chamomile; ½ jasmine, ¼ chamomile and ¼ mint; etc.).

An aroma (or scent) from the aromatic substances of the pouch 104 may enter the air intake 112. The aroma may then be provided to a subject using the CPAP device 102. For example, the aroma from the pouch 104 may enter the CPAP device 102 at the air intake 112. The aroma may then pass from the CPAP device 102 to the tube 108. The aroma may then pass through the tube 108 to the interface device 110. A subject may then receive the aroma from the interface device 110.

When the CPAP device 102 is turned on, air may be sucked in at the air intake 112. The breathability of the pouch 104 may allow the CPAP device 102 to suck in air even when the pouch 104 is covering the air intake 112 or a portion thereof. When air is sucked in, an aroma from the pouch 104 may be passed on to a user of the CPAP device 102. The pouch 104 may be removed for replacement and/or refill of the aromatic substances. A plurality of different aromatic substances may be used depending on the needs of the user.

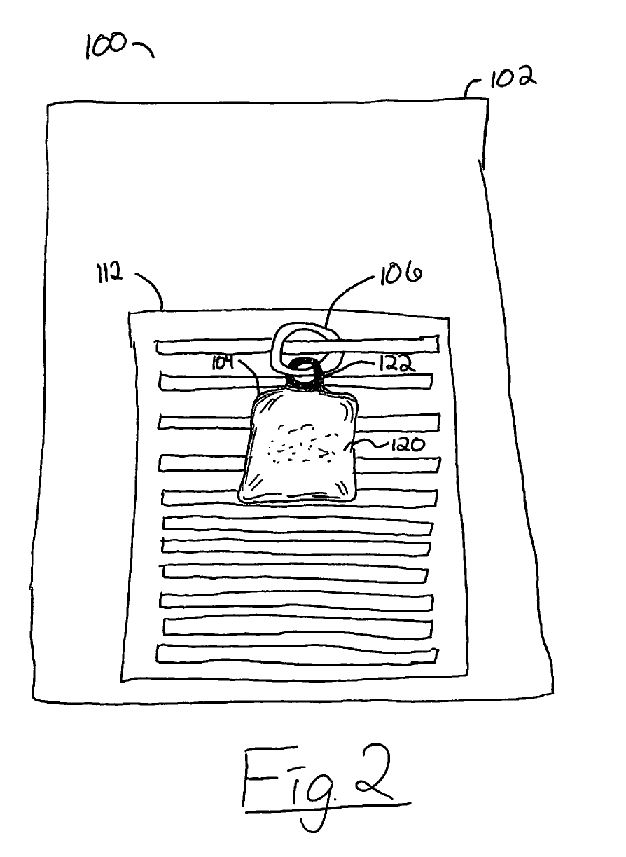

Referring to FIG. 2, a more detailed diagram of the apparatus 100 is shown. In particular, a more detailed diagram of the pouch 104, the coupling 106 and the air intake 112 is shown. The pouch 104 may comprise a first portion 120 and a second portion 122. The first portion 120 may be a cavity (or bag). The cavity 120 may be configured to hold (or contain) one or more aromatic substances (e.g., lavender, chamomile, jasmine, etc.). The second portion 122 may comprise a mechanism for securing the one or more objects in the cavity 120. The second portion 122 may be configured to secure the one or more objects (or aromatic substance(s)) inside the cavity 120.

The cavity 120 may be made from any breathable (or porous) material (e.g., organza, cotton, etc.) that allows for the dissemination of a scent. The pouch 104 and the coupling 106 may be made from the same material (e.g., organza). In one example, the second portion 122 may be made from the same material as the cavity 120 (e.g., organza, nylon, etc.). In another example, the pouch 104 may be made from a different material (e.g., organza) than the coupling 106.

In one embodiment, the second portion 122 may be the coupling 106. The second portion 122 may secure the one or more aromatic substance(s) inside the cavity 120 and attach the pouch 104 to the air intake 112. For example, the second portion 122 may be a noose lock. The second portion 122 may cinch the pouch 104 closed. Loose ends of the second portion 122 may attach the pouch 104 to the air intake 112 (e.g., in a knot), as shown in FIG. 2.

The pouch 104 may be cinched shut by any common design known in the art of securing a bag closed. For instance, two strings may be pulled in opposite directions such that the top of the pouch 104 is cinched closed. The pouch 104 may then be opened and closed multiple times. In another instance, the pouch 104 may be taped to the air intake 112 of the CPAP machine which also secures the aromatic substances inside the pouch 104.

In another example, the second portion 122 may attach to the coupling 106. The coupling 106 may then attach the pouch 104 to the air intake 112. Depending on the implementation, the coupling 106 may permanently attach to the air intake 112. In this example, the second portion 122 may attach and detach from the coupling 106. The second portion 122 and coupling 106 may form a locking connection (e.g., side release buckle, snap hook and ring, chord lock, hook and loop, snap fasteners, etc.).

In yet another example, the coupling 106 may be an adhesive (e.g., tape, glue, etc.). In this embodiment, the pouch 104 may be attached to the air intake 112 by the adhesive 106. The adhesive 106 may be removed from the air intake 112 so that a new pouch may be attached once the old one 104 has become ineffective. In general, the adhesive 106 may be removable from the air intake 112. The adhesive 106 may also permanently attach to the CPAP machine 102. Depending on the implementation, it may be necessary that the adhesive 106 permanently attach the pouch 104 to the air intake 112. In this instance, the aromatic substances may still be replaced inside the cavity 120 with fresh contents.

Referring to FIG. 3, an illustration of an apparatus 200 is shown. The apparatus 200 generally comprises a device 202, a vessel 204, and a device 206. The device 202 may be a ventilator device (e.g., a CPAP machine). The vessel 204 may be a pouch (or bag). The pouch 204 may be attached to the CPAP machine 202 by the device 206. The pouch 204 may be configured to hold (or contain) one or more objects (e.g., aromatic substances, aromatherapy products, etc.).

The CPAP machine 202 may have an air intake 208. Preferably, the pouch 204 may be attached to the CPAP machine 202 at the air intake 208. An aroma (or scent or smell) from the one or more aromatic substances may enter the air intake 208. The aroma may then be provided to a subject using the CPAP machine 202. The aroma from the one or more aromatic substances (or aromatherapy product(s)) may provide therapeutic benefits to the user.

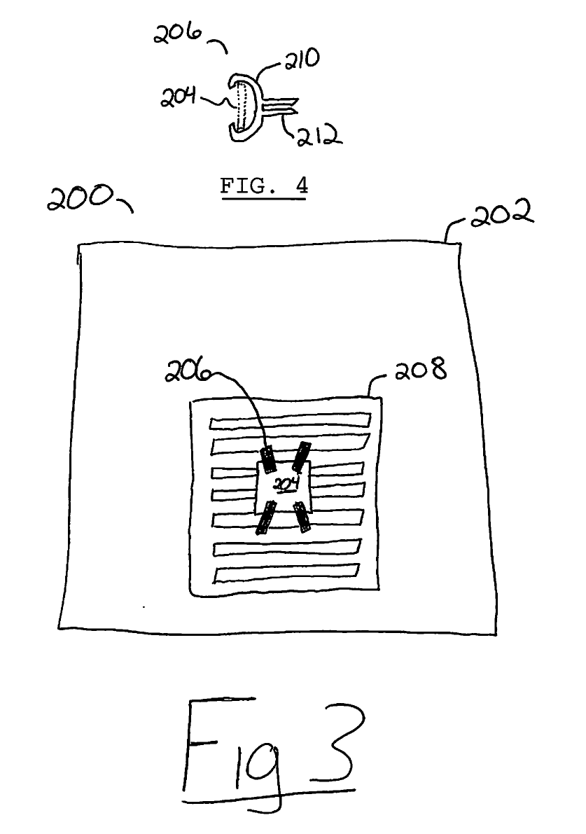

Referring to FIG. 4, a more detailed illustration of the device 206 is shown. The device 206 may comprise a first portion 210 and a second portion 212. The first portion 210 may be a receptacle (or cavity). The second portion 212 may be a coupler.

The receptacle 210 may be configured to embrace (or hold; or secure) the pouch 204. The coupler 212 may be configured to attach the device 206 to the CPAP machine 202. In one example, the coupler 212 may be a clip. It is known that there are many mechanisms for the coupler 212 to attach the device 206 to the CPAP machine 202. The example given is for illustrative purposes only. Any one of a plurality of mechanisms for attaching the device 206 to the CPAP machine 202 may be used.

The pouch 204 may further be configured to be removable from the receptacle 210. The pouch 204 may be made from a plurality of materials (e.g., organza, nylon, cotton, etc.). The device 206 may be made from a plurality of materials (e.g., metal, plastic, etc.). There is a plurality of materials that may be used to make either the pouch 204 or device 206. In one embodiment, the pouch 204 may be made from organza (or nylon) and the device 206 may be made from a plastic (e.g., HDPE, LDPE, PETE, PVC, etc.). In other embodiments, the device 206 may be made from metal.

In one embodiment, the second portion 212 may be made from the same material as the first portion 210 (e.g., plastic, metal, etc.). In another embodiment, the second portion may be made from a different material than the first portion 210.

The second portion 212 may attach the pouch 204 and first portion 210 to the air intake 208. Depending on the implementation, the second portion 212 may permanently attach to the air intake 208. For example, the second portion 212 may attach and detach from the first portion 210. The second portion 212 and first portion 210 may form a locking connection (e.g., side release buckle, snap hook and ring, chord lock, hook and loop, snap fasteners, etc.).

In another example, the second portion 212 may be an adhesive (e.g., tape, glue, etc.). In this embodiment, the device 206 may be attached to the air intake 208 by the adhesive 212. In general, the adhesive 212 may be removable from the air intake 208.

In another embodiment, the adhesive 212 may permanently attach to the CPAP machine 202. Depending on the implementation, it may be necessary that the adhesive 212 permanently attach the device 206 to the air intake 208. In this instance, the aromatic substances may still be replaced inside the first portion 210 with fresh contents.

The pouch 204 having aromatic substances may be held by the device 206, which may mechanically secure the pouch 204 to the CPAP machine 202. The device 206 use the first portion 210 to secure (or hold) the pouch and the second portion to attach the device 206 to the air intake 208. In one embodiment, the pouch 204 may be removable from the device 206 while the device 206 is (or is not) attached to the pouch 204. A user may then refill the contents of the pouch 204 and reattach the pouch 204 to the device 206.

The device 206 may be small in comparison to the overall size of the air intake 208. For example, the air intake 208 may be two times the size of the device 206. The device 206 may be of a universal design to allow it to be able to attach to a plurality of CPAP machines. The second portion 212 may use a plurality of attachment mechanisms known in the industry.

It will be apparent to those of ordinary skill in the art that the embodiments described herein, while illustrative, are not intended to so limit the invention or the scope of the appended claims. Those of ordinary skill will understand that various combinations or modifications of the embodiments presented herein may be made without departing from the spirit and scope of the present invention.

What is claimed is:

1. A system comprising:
a ventilator device;
a vessel that holds an aromatic substance; and
a coupler that mechanically connects said vessel to said ventilator device.

2. The system of claim 1, said ventilator device being a continuous positive airway pressure machine.

3. The system of claim 2, said coupler connects said vessel to an air intake of said ventilator device.

4. The system of claim 3, said aromatic substance comprising at least one of:
lavender;
jasmine;
mint; and
chamomile.

5. The system of claim 4, said vessel is a pouch.

6. The system of claim 5, said pouch comprises organza.

7. The system of claim 5, said pouch comprises nylon mesh.

8. The system of claim 5, said pouch comprises cotton.

9. The system of claim 3, said coupler is detachable from said air intake.

10. The system of claim 3, said coupler is fixed to said vessel.

11. The system of claim 3, said coupler has a first portion and a second portion.

12. The system of claim 11, said first portion is fixed to said vessel and said second portion is fixed to said air intake.

13. The system of claim 12, said first portion is detachable from said second portion.

14. A system comprising:
a ventilator device; and
a vessel having a first portion and a second portion, said vessel being connected to said ventilator device.

15. The system of claim 14, said ventilator device being a continuous positive airway pressure machine.

16. The system of claim 15, said first portion holds an aromatic substance.

17. The system of claim 16, said aromatic substance comprising at least one of:
lavender;
jasmine;
mint; and
chamomile.

18. The system of claim 15, said second portion securing said aromatic substance in said first portion.

19. The system of claim 18, said second portion being connected to an air intake of said ventilator device.

20. A system comprising:
a continuous positive airway pressure machine, said continuous positive airway pressure machine having an air intake;
a mesh pouch that holds an aromatic substance; and
a coupler that mechanically connects said mesh pouch to said air intake of said continuous positive airway pressure machine.
